(19) United States
(12) Patent Application Publication
(10) Pub. No.: US 2009/0281346 A1
(43) Pub. Date: Nov. 12, 2009

(54) METHOD FOR THE PRODUCTION OF CHIRAL AMINOCARBONYL COMPOUNDS

(75) Inventors: Benjamin List, Mulheim an der Ruhr (DE); Michael Stadler, Mulheim an der Ruhr (DE); Jung Woon Yang, Mulheim an der Ruhr (DE)

Correspondence Address: NORRIS, MCLAUGHLIN & MARCUS, PA, 875 THIRD AVENUE, 18TH FLOOR, NEW YORK, NY, 10022, US

(73) Assignee: STUDIENGESELLSCHAFT KOHLE MBH, Mülheim an der Ruhr (DE)

(21) Appl. No.: 12/373,955

(22) PCT Filed: Jul. 17, 2007
(86) PCT No.: PCT/DE07/01281

§ 371 Date: Jan. 15, 2009

(30) Foreign Application Priority Data
Jul. 19, 2006 (DE) 10 2006 033 362.4

(51) Int. Cl. C07C 271/18

(52) U.S. Cl. 560/24

(57) ABSTRACT

Disclosed is a method for producing aminocarbonyl compounds of the general formula (I)

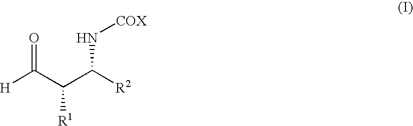

wherein
R¹ and R² can be identical or different and represents hydrogen, alkyl, alkenyl, alkynyl, or aryl,
X represents hydrogen, alkyl, alkenyl, alkynyl, aryl, or OR³, R³ representing hydrogen, alkyl, alkenyl, alkynyl, or aryl.

According to said method, an aldehyde of the general formula (II)

R¹CO (II)

wherein
R¹ has the meaning indicated above, is reacted with an imine of the general formula (III)

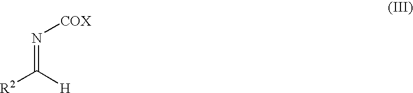

wherein R² and X have the meaning indicated above, in the presence of a catalyst.

Aminocarbonyles are obtained by means of catalyzed Mannich reactions with aldehydes. For example, if α-unbranched aldehydes are reacted with previously formed N-Boc imines in the presence of (S)-proline as a catalyst, the desired β-amino aldehydes are obtained at excellent yields, diastereoselectivities and enantioselectivities.

The present invention relates to a process for preparing aminocarbonyl compounds from aldehydes and imines in the presence of a catalyst.

The proline-catalyzed Mannich reaction between carbonyl compounds and imines (generated in situ) is a highly efficient and enantioselective method for synthesizing chiral nonracemic β-aminocarbonyl compounds (List et al. JACS 2000, 2002, Synlett 3003). This method in particular has been found to be useful in the synthesis of α- and β-amino acids, which are required for the synthesis of active pharmacological ingredients (Barbas JACS 2002, 2002, Hayashi Angew., Barbas 2006, Maruoka 2006). In this reaction, a distinction is drawn between two variants in which the imine is either generated in situ from aldehyde and amine (eq. 1) or has been preformed in a separate step (eq. 2) (scheme 1).

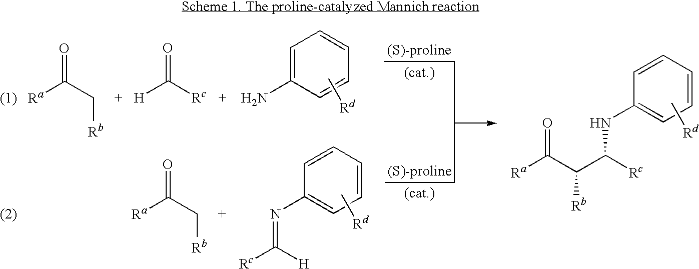

To date, it has been possible in this process to use exclusively imines which derive from aromatic amines (anilines). However, the removal of the aromatic radical from the nitrogen may be problematic. The typically used p-methoxyphenyl (PMP) group can, for example, be removed only by relatively drastic oxidative methods which often require toxic or expensive reagents, lead to by-products or cannot be performed on relatively sensitive substrates. The use of easily removable radicals on the nitrogen would therefore be desirable. Very valuable variants would, for example, be those in which the nitrogen is substituted in the form of a carbamate or amide. For example, benzyloxycarbonyl (Cbz or Z), tert-butoxycarbonyl (Boc) and fluorenylmethyloxycarbonyl groups (Fmoc) are used routinely and form the standard especially in the case of amino acids and in peptide synthesis. It was therefore an object of this invention to develop a proline-catalyzed Mannich reaction in which preformed imines or imines formed in situ are used, which are substituted by a readily eliminable group on the nitrogen.

The corresponding imines are already known in the literature or can be prepared in analogy to known processes. The customary synthesis comprises two simple stages (scheme 2). An aldehyde is thus first treated with an NH₂ carbamate and the sodium salt of an arylsulfonic acid. In this three-component reaction, the corresponding alkyloxycarbonyl-α-(arylsulfonyl)amine forms, which is reacted with base in a second step to give the desired imine.

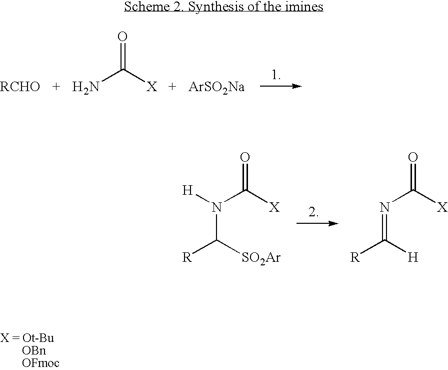

The present invention accordingly provides a process for preparing aminocarbonyl compounds of the general formula I

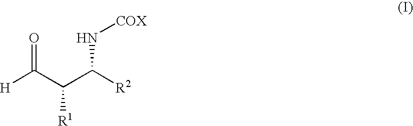

in which
R¹ and R² may be the same or different and are each hydrogen, alkyl, alkenyl, alkynyl or aryl,
X is hydrogen, alkyl, alkenyl, alkynyl or aryl, or is OR³ where R³ is hydrogen, alkyl, alkenyl, alkynyl or aryl,
in which an aldehyde of the general formula II

R′CO (II)

in which R¹ is as defined above
is reacted in the presence of a catalyst with an imine of the general formula III

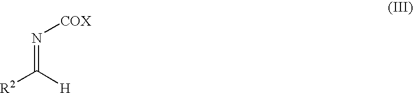

in which R² and X are each as defined above.

It has been found that, for example the imines of the above formula III are outstandingly suitable for proline-catalyzed Mannich reactions with aldehydes to obtain aminocarbonyls. When, for example, α-unbranched aldehydes are reacted with preformed N-Boc imines in the presence of (S)-proline as a catalyst, the desired β-amino aldehydes are formed in outstanding yields, diastereoselectivities and enantioselectivities.

To perform the process according to the invention, the reaction components are reacted in the presence of a catalyst. It is possible to use any desired catalyst which promotes the reaction between the aldehyde and the imine. When the reaction products to be prepared are chiral aminocarbonyls, preference is given to using asymmetric catalysts, especially asymmetric organic catalysts. Particularly suitable catalysts have been found to be those which contain one or more heteroatoms, for example nitrogen, oxygen, sulfur or phosphorus, nitrogen being a preferred heteroatom. Oxygen- or sulfur-containing catalysts may, for example, be alcohol and thiols, while phosphorus-containing catalysts are generally phosphines. Catalysts with one or more nitrogen atoms in the molecule may be primary or secondary amines or nitrogen-containing polymers. Preferred amines have a structure with the general formula IV

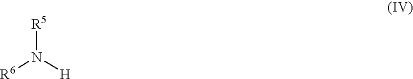

in which
R⁵ and R⁶ may be the same or different and are selected from hydrogen, hydrocarbons, especially alkyl, alkenyl, alkynyl, aryl or alkylaryl, each of which may have suitable substituents or one or more heteroatoms in the radical, or R⁵ and R⁶ together form a ring structure which, in addition to the nitrogen atom in the formula IV, may optionally contain a further heteroatom. When R⁵ and R⁶ are bonded to one another, they may, for example, form a five- or six-membered alicyclic or aromatic ring, i.e. R⁵ and R⁶ may be unsubstituted or substituted cyclopentyl, cyclohexyl, pyrrolidinyl, piperidinyl, morpholinyl, pyrrolyl, pyridinyl, pyrimidinyl, imidazolyl or the like. Preferred compounds are those in which R⁵ and R⁶ are each independently selected from methyl, ethyl, propyl, butyl, cyclopentyl, cyclohexyl, cyclooctyl, phenyl, naphthyl, benzyl and trimethylsilyl or the like, such that a 3- to 15-membered, optionally substituted cyclic radical with the general formula V is formed

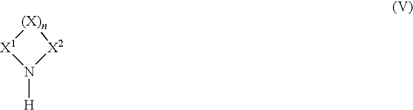

in which n is 0 or 1 and X is a radical having up to 50 atoms which is selected from the group of the substituted and unsubstituted alkylenes which may also contain heteroatoms, and X¹ and X² are each independently an unsubstituted or substituted methylene group. Examples of the secondary amines of the formula V are compounds of the general formula VI

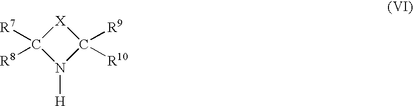

in which
R⁷, R⁸, R⁹ and R¹⁰ may be the same or different and are each independently selected from hydrogen, OH, SH, carboxyl, amino, mono-C₁-C₂₄-alkylamino, di-C₁-C₂₄-alkylamino, mono-C₅-C₂₄-arylamino, di-C₅-C₂₄-arylamino, di-N-substituted C₁-C₂₄-alkyl-C₅-C₂₄-arylamino, C₂-C₂₄-alkylamido, C₆-C₂₄-arylamido, imino, C₂-C₂₄-alkylimino, C₆-C₂₄-arylimino, nitro, nitroso, C₁-C₂₄-alkoxy, C₅-C₂₄-aryloxy, C₆-C₂₄-aralkyloxy, C₂C₂₄-alkylcarbonyl, C₆-C₂₄-arylcarbonyl, C₂-C₂₄-alkylcarbonyloxy, C₆-C₂₄-arylcarbonyloxy, C₂-C₂₀-alkoxycarbonyl, C₆-C₂₄-aryloxycarbonyl, halocarbonyl, carbamoyl, monosubstituted C₁-C₂₄-alkylcarbamoyl, di-N-substituted C₁-C₂₄-alkylcarbamoyl, di-N-substituted N—C₁-C₂₄-alkyl-N—C₅-C₂₄-aryl-carbamoyl, monosubstituted C₅-C₂₄-arylcarbamoyl, di-N-substituted C₅-C₂₄-aryl-carbamoyl, thiocarbamoyl, monosubstituted C₁-C₂₄-alkylthiocarbamoyl, di-N-substituted C₁-C₂₄-alkylthiocarbamoyl, di-N-substituted N—C₁-C₂₄-alkyl-N—C₅-C₂₄-aryl-thiocarbamoyl, monosubstituted C₅-C₂₄-arylthiocarbamoyl, di-N-substituted C₅-C₂₄-arylthiocarbamoyl, carbamido, formyl, thioformyl, sulfo, sulfonato, C₁-C₂₄-alkylthio, C₅-C₂₄-arylthio, C₁-C₂₄-alkyl-substituted C₁-C₂₄-alkyl, C₁-C₂₄-heteroalkyl, substituted C₁-C₂₄-heteroalkyl, C₅-C₂₄-aryl, substituted C₅-C₂₄-aryl, C₅-C₂₄-heteroaryl, substituted C₅-C₂₄-heteroaryl, C₂-C₂₄-aralkyl, substituted C₂-C₂₄-aralkyl, C₂-C₂₄-heteroaralkyl and C₂-C₂₄-heteroaralkyl, or R⁷, and R⁸ and/or R⁹ and R¹⁰ together form an ═O radical.

X may, for example, be a —(CR¹¹R¹²)—(X³)_{q}—(CR¹³R¹⁴)ₜ group, such that the amine is a compound of the general formula VII

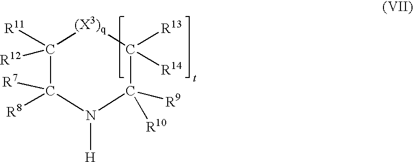

in which
X³ is O, S, NH, NR¹⁵ or CR¹⁶R¹⁷, q is 0 or 1, t is 0 or 1, and R¹¹, R¹², R¹³, R¹⁴, R¹⁶ and R¹⁷ are each independently selected from hydrogen, OH, SH, carboxyl, amino, mono-C₁-C₂₄-alkylamino, di-C₁-C₂₄-alkylamino, mono-C₅-C₂₄-arylamino, di-C₅-C₂₄-arylamino, di-N-substituted C₁-C₂₄-alkyl-C₅-C₂₄-arylamino, C₂-C₂₄-alkylamido, C₆-C₂₄-arylamido, imino, C₂-C₂₄-alkylimino, C₆-C₂₄-arylimino, nitro, nitroso, C₁-C₂₄-alkoxy, C₅-C₂₄-aryloxy, C₆-C₂₄-aralkyloxy, C₂-C₂₄-alkylcarbonyl, C₆-C₂₄-arylcarbonyl, C₂-C₂₄-alkylcarbonyloxy, C₆-C₂₄-arylcarbonyloxy, C₂-C₂₀-alkoxycarbonyl, C₆-C₂₄-aryloxycarbonyl, halocarbonyl, carbamoyl, monosubstituted C₁-C₂₄-alkylcarbamoyl, di-N-substituted C₁-C₂₄-alkylcarbamoyl, di-N-substituted N—C₁-C₂₄-alkyl-N—C₅-C₂₄-aryl-carbamoyl, monosubstituted C₅-C₂₄-arylcarbamoyl, di-N-substituted C₅-C₂₄-aryl-carbamoyl, thiocarbamoyl, monosubstituted C₁-C₂₄-alkylthiocarbamoyl, di-N-substituted C₁-C₂₄-alkylthiocarbamoyl, di-N-substituted N—C₁-C₂₄-alkyl-N—C₅-C₂₄-aryl-thiocarbamoyl, monosubstituted C₅-C₂₄-arylthiocarbamoyl, di-N-substituted C₅-C₂₄-arylthiocarbamoyl, carbamido, formyl, thioformyl, sulfo, sulfonato, C₁-C₂₄-alkylthio, C₅-C₂₄-arylthio, C₁-C₂₄-alkyl, substituted C₁-C₂₄-alkyl, C₁-C₂₄-heteroalkyl, substituted C₁-C₂₄-heteroalkyl, C₅-C₂₄-aryl, substituted C₅-C₂₄-aryl, C₅-C₂₄-heteroaryl, substituted C₅-C₂₄-heteroaryl, C₆-C₂₄-aralkyl, substituted C₆-C₂₄-aralkyl, C₂-C₂₄-heteroaralkyl and substituted C₂-C₂₄-heteroaralkyl, or R¹¹ and R¹², and/or R¹³ and R¹⁴, together form an ═O radical, and R¹⁵ is selected from substituted or unsubstituted, saturated or unsaturated hydrocarbons having from 1 to 12 carbon atoms, which may also contain one or more heteroatoms.

Preference is given to catalysts of the formula VI in which q is 0, t is 1 and at least one of the R⁷ to R¹⁰ radicals is an acidic substituent, such as a carboxyl group; in such a configuration, the compound of the formula VII is proline or substituted proline. A suitable catalyst is L-proline itself, a compound known from the literature, which corresponds to the compound of the formula VII when R⁷ to R⁹ and R¹¹ to R¹⁴ are each hydrogen and R¹⁰ is β-carboxyl.

A further group of catalysts used with preference is that of compounds in which q is 1, X³ is NR¹⁵, t is 0, R⁷ and R⁹ are each hydrogen and R⁸ is CR¹⁸R¹⁹R²⁰, such that the secondary amine is a compound of the general formula VIIIA or VIIIB

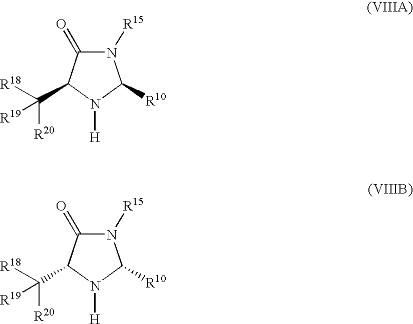

in which
R¹⁰ is as defined above and is preferably an -(L)ₘ-CR¹⁹R²⁰R²³ group in which m is 0 or 1, L is C₁-C₆-alkylene and R²¹, R²² and R²⁴ are each hydrocarbons having from 1 to 12 carbon atoms. The substituents R⁸ are preferably those in which m is 0, and R²¹, R²² and R²³ are each C₁-C₁₂-alkyl. More preferably, R²¹, R²² and R²³ are each C₁-C₆-alkyl, especially methyl, and so R⁸ is a t-butyl group.
R¹⁵ is selected from substituted and unsubstituted hydrocarbons having from 1 to 12 carbon atoms, for example alkyl, alkenyl, alkynyl, aryl, alkaryl, aralkyl, etc., which may contain one or more heteroatoms. R¹⁵ preferably represents hydrocarbons having from 1 to 12 carbon atoms, such as C₁-C₁₂-alkyl, preference being given to C₁-C₆-alkyl, such as methyl, ethyl, propyl, butyl, pentyl or hexyl.
R¹⁸ and R¹⁹ are each independently selected from hydrogen, halogen, hydroxyl, substituted or unsubstituted hydrocarbons having from 1 to 12 carbon atoms, which may contain one or more heteroatoms. R¹⁸ and R¹⁹ are preferably each hydrogen or hydrocarbon having from 1 to 12 carbon atoms, particular preference being given to R¹⁸ and R¹⁹.
R²⁰ may be a cycle which may have from 1 to 4 substituents and from 0 to 3 heteroatoms selected from N, O and S. In a preferred embodiment, R²⁰ is a monocyclic aryl or heteroaryl having up to 4 substituents which are selected from halogen, hydroxyl and hydrocarbon having from 1 to 12 carbon atoms. R²⁰ is more preferably a phenyl group which may have 1 or 2 substituents, such as halogen, hydroxyl or C₁-C₆-alkyl, where R²⁰ is most preferably an unsubstituted phenyl group.

Any of the above-described compounds may also be used in the form of the acid addition salts, in which case the addition salt per se may be used or it may form in the course of the reaction.

Particularly preferred catalysts are shown below:

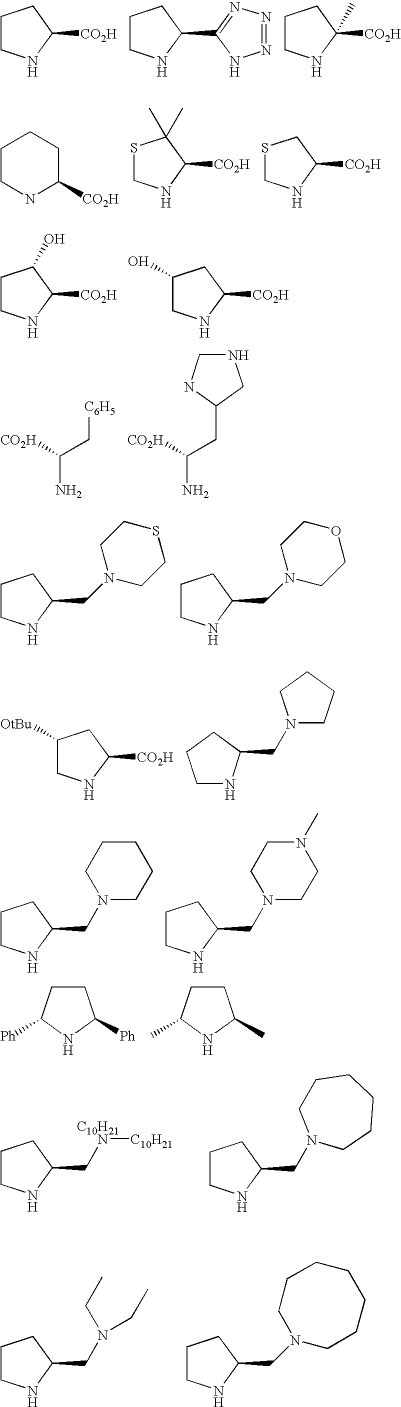

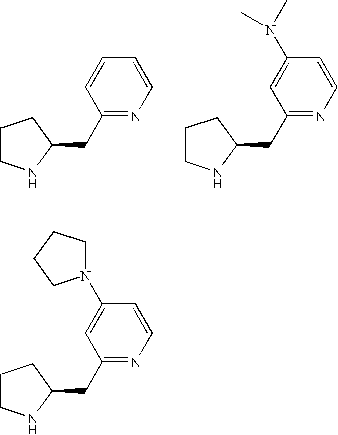

The catalyst is typically used in an amount of from 0.1 to 200 mol %, preferably from 1 to 30 mol %, based on the starting compounds.

The imines of the general formula III may be used for the process according to the invention directly or in the form of their pre-stages, such that the imine is formed in situ during the reaction.

The term “alkyl” used means a linear, branched or cyclic hydrocarbon radical which has typically from 1 to 30, preferably from 1 to 24 carbon atoms and especially from 1 to 6 carbon atoms, such as methyl, ethyl, n-propyl, isopropyl, n-butyl, isobutyl, t-butyl, octyl, decyl, etc., but also cycloalkyl groups such as cyclopentyl, cyclohexyl, etc. The hydrocarbon radicals preferably have from 1 to 18, especially from 1 to 12 carbon atoms.

In the context of the present invention, “alkenyl” means an unsaturated, linear, branched, or cyclic hydrocarbon radical which has one or more double bonds and typically between 2 and 30, preferably from 2 to 24 and especially from 2 to 6 carbon atoms, such as ethenyl, n-propenyl, isopropenyl, n-butenyl, isobutenyl, pentenyl, hexenyl, octenyl, decenyl, etc., but also cycloalkenyl groups such as cyclopentenyl, cyclohexenyl, etc.

In the context of the present invention, “alkynyl” means an unsaturated, linear, branched, or cyclic hydrocarbon radical which has one or more triple bonds and typically between 2 and 30, preferably from 2 to 24 and especially from 2 to 6 carbon atoms, such as ethynyl, n-propynyl, isopropynyl, n-butynyl, isobutynyl, pentynyl, hexynyl, octynyl, decynyl, etc., but also cycloalkynyl groups such as cyclopentynyl, cyclohexynyl, etc.

In the context of the present invention, the aryl groups used are aromatic ring systems having from 5 to 30 carbon atoms and optionally heteroatoms such as N, O, S, P, Si in the ring, where the rings may be single or multiple ring systems, for example fused ring systems, or rings bonded to one another via single bonds or multiple bonds. Examples of aromatic rings are phenyl, naphthyl, biphenyl, diphenyl ether, diphenylamine, benzophenone and the like. Substituted aryl groups have one or more substituents. Examples of heteroalkyl groups are alkoxyaryl, alkylsulfanyl-substituted alkyl, N-alkylated aminoalkyl and the like. Examples of heteroaryl substituents are pyrrolyl, pyrrolidinyl, pyridinyl, quinolinyl, indolyl, pyrimidinyl, imidazolyl, 1,2,4-triazolyl, tetrazolyl and the like. Examples of heteroatom-containing alicyclic groups include pyrrolidino, morpholino, piperazino, piperidino, etc.

Useful substituents that the aforementioned groups may have include OH, F, Cl, Br, J, CN, NO₂, NO, SO₂, SO₃⁻, amino, —COOH, —COO(C₁-C₆-alkyl), mono- and di-(C₁-C₂₄-alkyl)-substituted amino, mono- and di-(C₅-C₂₀-aryl)-substituted amino, imino, which may in turn be substituted, for example C₁-C₆-alkyl, aryl and phenyl. Especially the cyclic radicals may also have C₁-C₆-alkyl groups as substituents.

The process according to the invention is preferably performed in solution. To this end, at least one of the starting substances or the catalyst is dissolved in a suitable solvent; the further components are added as pure substances or in solution. The solvents used may be any organic solvents which are inert toward the reaction components and do not intervene in the reaction. Examples of suitable solvents are pentane, hexane, heptane, octane, petroleum ether, toluene, xylenes, ethyl acetate, tetrahydrofuran, diethyl ether, methyl tert-butyl ether, 1,4-dioxane, methylene chloride, chloroform, carbon tetrachloride, dimethyl sulfoxide, dimethylformamide, N-methylpyrrolidinone, acetonitrile, methanol, ethanol, dioxane, sulfolane, 1,2-dichloroethane, poly(ethylene glycol) having a molecular weight between 200 and 1450, preferably between 200 and 600, ionic liquids, water and any desired mixtures of the above, preference being given to organic solvents.

The process according to the invention can be performed within wide temperature ranges; the reaction temperature is typically between −20° C. and 50° C. The reaction time is between 1 hour and 24 hours. The resulting reaction product can typically be isolated from the reaction mixture and purified. In one possible embodiment, the reaction mixture is added to water and then extracted with an organic solvent.

### EXAMPLES

### General Method

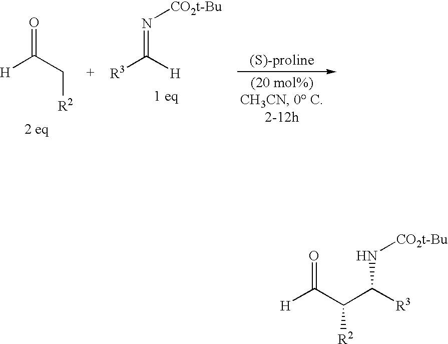

The N-Boc imine (0.5 mmol) was dissolved in dry acetonitrile (5 ml) and admixed at 0° C. with the aldehyde (1 mmol, 2 equiv.) and with (L)- or (D)-proline (0.1 mmol, 20 mol %). After 2-12 h at 0° C., the pure product precipitates out and can be isolated by filtration and washing with cold hexane. If the product does not precipitate out, or does so only incompletely, the reaction mixture is added to water and extracted with ether. The combined organic phases are dried and concentrated, and the pure product is isolated by trituration with cold hexane. The enantiomeric purities were determined by means of HPLC of the crude mixture (before the crystallization) (see scheme 3).

Scheme 3. Proline-catalyzed Mannich reaction between
aldehydes and N-Boc imines

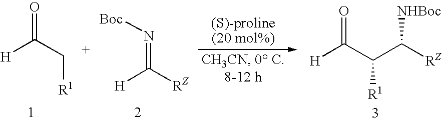

| Ex- | | Yield | | |
|---|---|---|---|---|
| ample | Product | [%] | de | ee |
| (1) | 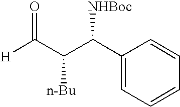 3a | 84 | >99:1 | >99:1 |
| (2) | 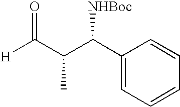 3b | 91 | >99:1 | >99:1 |
| (3) | 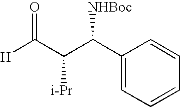 3c | 88 | >99:1 | >99:1 |
| (4) | 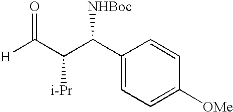 3d | 80 | >99:1 | >99:1 |
| (5) | 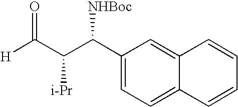 3e | 82 | >99:1 | >99:1 |
| (6) | 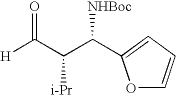 3f | 74 | >99:1 | 98:2 |
| (7) | 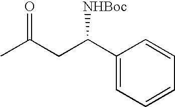 3g | 73 | — | >99:1 |

### Use of Acetaldehyde

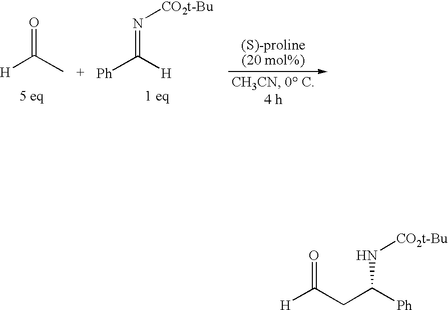

The N-Boc imine (287.4 mg, 1.4 mmol) was dissolved in 9.5 ml of a 0.74M solution of acetaldehyde (5 eq.) in dry acetonitrile, cooled to 0° C. and then admixed with (L)-proline (32.2 mg, 0.28 mmol, 20 mol %). After 4 h at 0° C., the reaction mixture was added to water and extracted three times with diethyl ether. The combined organic phases were washed once with saturated aqueous sodium chloride solution and dried over-MgSO₄. The product was purified by column chromatography on silica gel using ethyl acetate/hexane (first 10/90, then 20/80, vol/vol) as the eluent. The product is obtained in 52% yield. The enantiomeric ratio of the product was determined by means of gas chromatography to be >99:1.

1. A process for preparing aminocarbonyl compounds of the formula I: 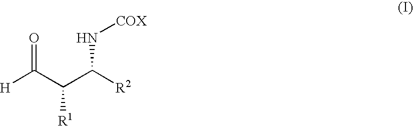
in which
R¹ and R² may be the same or different and are each hydrogen, alkyl, alkenyl, alkynyl or aryl,
X is hydrogen, alkyl, alkenyl, alkynyl or aryl, or is OR³ where R³ is hydrogen, alkyl, alkenyl, alkynyl or aryl,
comprising reacting an aldehyde of the formula II:
R¹CO (II):
in which R¹ is as defined above
in the presence of a catalyst with an imine of the general formula III: 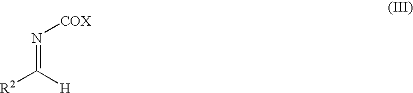
in which R² and X are each as defined above.

2. The process as claimed in claim 1, wherein the catalyst is selected from the group consisting of asymmetric organic catalysts.

3. The process as claimed in claim 2, wherein the catalyst is a chiral amino acid.

4. The process as claimed in claim 1, wherein X is a OR³ group in which R³ is an alkyl radical having from 1 to 6 carbon atoms.
